(19) United States
(12) Patent Application Publication
(10) Pub. No.: US 2006/0025765 A1
(43) Pub. Date: Feb. 2, 2006

(54) Electrosurgical systems and methods

(76) Inventors: Jaime Landman, St. Louis, MO (US); Manu Mohan Sachdev, Newark, DE (US); Lauren Elizabeth Hueser, Brighton, MA (US); Jaeyun Kim, Clayton, MO (US)

Correspondence Address: HARNESS, DICKEY, & PIERCE, P.L.C, 7700 BONHOMME, STE 400, ST. LOUIS, MO, 63105, US

(21) Appl. No.: 11/194,122

(22) Filed: Jul. 29, 2005

Related U.S. Application Data
(60) Provisional application No. 60/592,821, filed on Jul. 30, 2004.

(51) Int. Cl. A61B 18/14

(52) U.S. Cl. 606/48; 606/50

(57) ABSTRACT

An electrosurgical device includes a plurality of electrodes for forming a plurality of bipolar circuits usable for affecting a patient's tissue during a surgical operation. At least one of said electrodes is operable as an active electrode in one of said bipolar circuits and as a return electrode in another one of said bipolar circuits.

### CROSS-REFERENCE TO RELATED APPLICATIONS

This application claims the benefit of U.S. Provisional Application No. 60/592,281, filed on Jul. 30, 2004. The disclosure(s) of the above application(s) is (are) incorporated herein by reference.

### FIELD OF INVENTION

The present invention generally relates to electrosurgical systems including electrosurgical devices having a plurality of electrodes.

### BACKGROUND OF INVENTION

The combination of engineering and medicine has allowed doctors to take advantage of technology to offer their patients more effective and less invasive treatments, especially in various areas of surgery. Electrosurgery is one of these areas developed through technology. Electrosurgery is a surgical method in which current/voltage is passed through tissue to induce a thermo physical effect. Depending on needs of the doctor and/or the treatment, the thermo physical effect on the tissue can be understood to include cutting, cauterizing, coagulating, etc.

Generally, two electrosurgical devices exist for various types of procedures: a monopolar device and a bipolar device. The monopolar device includes a single active electrode and a dispersive (neutral) electrode (usually a pad placed on the patent's body). The bipolar device includes an active electrode and a neutral electrode fixedly positioned in close proximity. When either device is used, an activity site exists between the active electrode and its respective neutral electrode. When the active electrode is energized, the current/voltage induces a thermo physical effect to at least a portion of the tissue within the activity site.

As recognized by the inventors hereof, while electrosurgery has been an advantage in some treatments, the negative consequences of the existing devices have limited the use of electrosurgery. The first of several negative consequences is damage caused to conductive tissue in close proximity to the activity site. Another negative consequence is that for greater amounts of tissue within the activity site, existing devices generally affect the tissue inconsistently.

### SUMMARY OF INVENTION

The inventors hereof have succeeded at designing electrosurgical devices capable of a more precise and consistent thermo effect on various amounts of tissue.

According to one aspect of the invention, an electrosurgical device includes a plurality of electrodes for forming a plurality of bipolar circuits usable for affecting a patient's tissue during a surgical operation. At least one of said electrodes is operable as an active electrode in one of said bipolar circuits and as a return electrode in another one of said bipolar circuits.

According to another aspect of the invention, an electrosurgical device includes a plurality of electrodes, including a plurality of adjacent electrode pairs, and a controller for selectively activating the adjacent electrode pairs to incrementally affect tissue including a first tissue portion and a second tissue portion. Activating a first electrode pair affects the first tissue portion, and activating a second electrode pair affect the second tissue portion.

According to yet another aspect of the invention, a method for applying electrosurgical energy to tissue with an electrosurgical device having a plurality of electrodes for forming of a plurality of bipolar circuits, the method includes using at least one of said electrodes as an active electrode in one of said bipolar circuits, and then as a return electrode in another one of said bipolar circuits.

According to another aspect of the invention, a method for incrementally affecting tissue including a first tissue portion and a second tissue portion with an electrosurgical device having a plurality of electrodes including a plurality of adjacent electrode pairs, the method includes activating a first electrode pair to affect the first tissue portion, and activating a second electrode pair to affect the second tissue portion.

According to yet another aspect of the invention, an electrosurgical device includes a plurality of electrodes arranged in a linear array for forming a plurality of bipolar circuits each usable for affecting a patient's tissue during a surgical operation.

According to another aspect of the invention, an electrosurgical device includes a plurality of electrodes. A section of each electrode is intended to make contact with a patient's tissue. The section includes an electrically insulative portion and an electrically conductive portion.

According to still another aspect of the invention, an electrode deployment device for an electrosurgical device includes a plurality of electrodes arranged in a linear array. The deployment device is capable of moving the electrodes between a collapsed position in which the electrodes are generally parallel with one another, and an expanded position in which the electrodes are fanned out.

According to yet another aspect of the invention, an electrosurgical device includes a plurality of electrodes arranged in a linear array in which electrode length increases from the innermost electrode to the outermost electrodes within the linear array.

Further aspects of the present invention will be in part apparent and in part pointed out below. It should be understood that various aspects of the invention may be implemented individually or in combination with one another. It should also be understood that the detailed description and drawings, while indicating certain exemplary embodiments of the invention, are intended for purposes of illustration only and should not be construed as limiting the scope of the invention.

### BRIEF DESCRIPTION OF THE DRAWINGS

The present invention will become more fully understood from the detailed description and the accompanying drawings, wherein:

FIG. 1 is an illustration of an exemplary system including an electrosurgical unit having a plurality of electrodes, a controller, and a generator according to one embodiment of the invention;

FIG. 2 is a perspective view of the end effector of the electrosurgical unit shown in FIG. 1 with the electrodes in an expanded position;

FIG. 3 is another perspective view of an end effector of an electrosurgical unit according to one embodiment of the invention;

FIG. 4 is perspective view of an end effector having a plurality of electrodes being used to affect tissue during a surgical procedure according to one embodiment of the invention;

FIG. 5 illustrates various exemplary electrode deployed configurations, cross-sectional shapes, and tip shapes that may be used for an electrosurgical unit according to embodiments of the present invention;

FIG. 6 is a upper plan view of an end effector for an electrosurgical unit according to one embodiment of the invention;

FIG. 7 is an upper plan view of an end effector showing the electrodes in an expanded or deployed position and providing exemplary dimensions according to one embodiment of the invention;

FIG. 8 is another upper plan view of the end effector shown in FIG. 7 but showing the electrodes in a collapsed or stowed position and also providing exemplary dimensions according to one embodiment of the invention;

FIG. 9 is a perspective view of an electrode that may be used in an electrosurgical unit according to one embodiment of the invention;

FIG. 10 is an upper plan view of the electrode shown in FIG. 9;

FIG. 11 is a side elevation view of the electrode shown in FIG. 9;

FIG. 12 is an end elevation view showing the tip of the electrode shown in FIG. 9;

FIG. 13 is a perspective view of an electrosurgical unit according to one embodiment of the invention;

FIG. 14 is an upper plan view of the electrosurgical unit shown in FIG. 13;

FIG. 15 is a side elevation view of the electrosurgical unit shown in FIG. 13;

FIG. 16 is an end elevation view of the electrosurgical unit shown in FIG. 13;

FIG. 17 is an exploded view of a back portion of the electrosurgical unit shown in FIG. 13;

FIG. 18 is side cross-sectional views of a back portion of the electrosurgical unit shown in FIG. 13 according to one embodiment of the invention;

FIG. 19 is an exploded plan view of an end effector for an electrosurgical unit according to one embodiment of the invention;

FIG. 20 is a perspective view of the handle shown in FIG. 13;

FIG. 21 is an upper plan view of the handle shown in FIG. 20;

FIG. 22 is a side elevation view of the handle shown in FIG. 20;

FIG. 23 is an end elevation view of the handle shown in FIG. 20;

FIG. 24 is a lower plan view of the handle shown in FIG. 20;

FIG. 25 is a perspective view of the body shown in FIG. 13;

FIG. 26 is an upper plan view of the body shown in FIG. 25;

FIG. 27 is a cross-sectional view of the body taken along the plane A-A in FIG. 26;

FIG. 28 is a side elevation view of the body shown in FIG. 25;

FIG. 29 is a lower plan view of the body shown in FIG. 25;

FIG. 30 is an end elevation view of the body shown in FIG. 25;

FIG. 31 is a perspective view of the trigger shown in FIG. 13;

FIG. 32 is an upper plan view of the trigger shown in FIG. 31;

FIG. 33 is a side elevation view of the trigger shown in FIG. 31;

FIG. 34 is an end elevation view of the trigger shown in FIG. 31;

FIG. 35 is a perspective view of the secondary rod shown in FIGS. 17 and 18;

FIG. 36 is an upper plan view of the secondary rod shown in FIG. 35;

FIG. 37 is a cross-sectional view of the secondary rod taken along the plane B-B in FIG. 36;

FIG. 38 is a side elevation view of the secondary rod shown in FIG. 35;

FIG. 39 is an end elevation view of the secondary rod shown in FIG. 35;

FIG. 40 is a perspective view of the main rod shown in FIGS. 17 and 18;

FIG. 41 is an upper plan view of the main rod shown in FIG. 40;

FIG. 42 is a side elevation view of the main rod shown in FIG. 40;

FIG. 43 is an elevation view of the portion designated as “C” in FIG. 46;

FIG. 44 is a perspective view of the shaft shown in FIG. 13;

FIG. 45 is an upper plan view of the shaft shown in FIG. 44;

FIG. 46 is a cross-sectional view of the shaft taken along the plane D-D in FIG. 45;

FIG. 47 is an elevation view of the portion designated as “E” in FIG. 46;

FIG. 48 is a side elevation view of the shaft shown in FIG. 44;

FIG. 49 is an end elevation view of the shaft shown in FIG. 44;

FIG. 50 is a cross-sectional view showing the main rod (FIG. 40) positioned within the shaft (FIG. 44);

FIG. 51 is a perspective view of the sliding bar shown in FIG. 19;

FIG. 52 is an upper plan view of the sliding bar shown in FIG. 51;

FIG. 53 is a side elevation view of the sliding bar shown in FIG. 51;

FIG. 54 is an end elevation view of the sliding bar shown in FIG. 51;

FIG. 55 is a perspective view of the stationary bar shown in FIG. 19;

FIG. 56 is an upper plan view of the stationary bar shown in FIG. 55;

FIG. 57 is a side elevation view of the stationary bar shown in FIG. 55;

FIG. 58 is an end elevation view of the stationary bar shown in FIG. 55;

FIG. 59 is an exemplary circuit diagram for an electrosurgical unit including a plurality of electrodes which can be controlled in accordance with a timed sequence according to one embodiment of the invention;

FIG. 60 is a diagram of the exemplary controller shown in FIG. 59;

FIG. 61 is a flow diagram of an exemplary method for controlling voltage and current applied to electrodes in an electrosurgical device in accordance with a timed sequence;

FIG. 62 form a flow diagram of an exemplary method for controlling voltage and current applied to electrodes in an electrosurgical device in accordance with impedance caused by the tissue through which electrical current is flowing.

FIG. 63 is an exemplary circuit diagram for an electrosurgical unit including a plurality of electrodes which can be controlled in accordance with impedance feedback according to one embodiment of the invention.

The various exemplary dimensions, shapes, and materials set forth in the figures are for purposes of illustration only and are in no way intended to limit the invention, its application, or uses.

### DETAILED DESCRIPTION OF THE EXEMPLARY EMBODIMENTS

The following description of the exemplary embodiments is merely exemplary in nature and is in no way intended to limit the invention, its application, or uses.

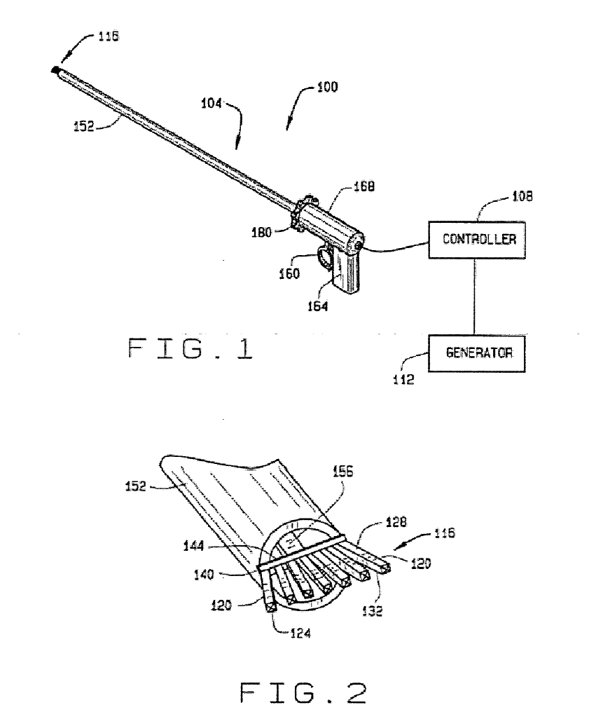

An electrosurgical system according to one exemplary embodiment of the invention is indicated generally in FIG. 1 by reference number 100. As shown, the system 100 includes an electrosurgical unit 104 capable of applying electrosurgical energy (e.g., RF energy) for cutting, cauterizing, and/or coagulating a patient's tissue. As described in more detail below, the electrosurgical unit 104 includes a plurality of electrodes 120 capable of forming a plurality of bipolar circuits. These bipolar circuits can each be formed by a corresponding pairs of electrodes and a patient's tissue in electrical contact with the electrode pair.

The system 100 can also include a controller 108 and a generator 112. Generally, the controller 108 can be used to control the operation of the electrosurgical unit 104, while the generator 112 provides the electrical power for the electrosurgical unit 104, and the ground for the electrodes 120 and bipolar circuits formed therewith.

Either or both the controller 108 and/or the generator 112 can include an electrical cord configured for connecting with a standard wall outlet. Alternatively, other power sources (e.g., batteries, etc.) can be used for the controller 108 and/or the generator 112. Either or both the controller 108 and/or the generator 112 can be integrated into the electrosurgical unit 104. For example, controller 108 may be mounted into a handle 164 and/or a body 168.

In one implementation (described in more detail below), the electrodes 120 are configured to fire sequentially after the tissue has been penetrated with the end effector 116 of the electrosurgical unit 104. In which case, current can pass from a first electrode (active) to a second electrode (passive) in one firing sequence. In the next firing sequence, the second electrode becomes active and the third becomes passive. This set of events can continue until each of the six bipolar electrode sets have fired. This sequential firing can be controlled by a switching interface. This interface can include relays to switch the relatively high currents and voltages produced by the generator 112. The switching can be controlled by the controller 108. By using multiple sets of bipolar electrodes, the electrosurgical device 104 can effectively cauterize, cut, coagulate or otherwise affect multiple tissue layers over a relatively large surface area in comparison to that of bipolar needles and other existing bipolar and monopolar devices.

As shown in FIGS. 2-3 and 6-8, the electrosurgical unit 104 includes an end effector 116 having seven electrodes 120. Alternatively, other suitable numbers of electrodes can be used depending on the particular application (e.g., type of surgery, size of trocar or access mechanism, type of tissue, etc.) intended for the electrosurgical unit 104. For example, FIGS. 4 and 5 illustrate exemplary implementations in which six electrodes are used.

According to one aspect of the invention, the electrodes 120 are arranged linearly to form a linear array. Alternatively, the electrodes can be arranged differently (e.g., in a rectangular array, circular array, semi-circular array, etc.) depending on the particular application or surgical procedure intended for the electrosurgical unit 104.

A wide range of cross-sectional shapes can be used for the electrodes 120 including those exemplary cross-sectional shapes shown in FIG. 5. Electrode cross-sectional shape can vary depending on the particular application or surgical procedure intended for the electrosurgical unit 104. Various criteria can be used to determine a suitable electrode cross-sectional shape including ability to provide a high current density between the electrodes, pressure in the tissue between the electrodes, and a low degree of nonspecific tissue damage.

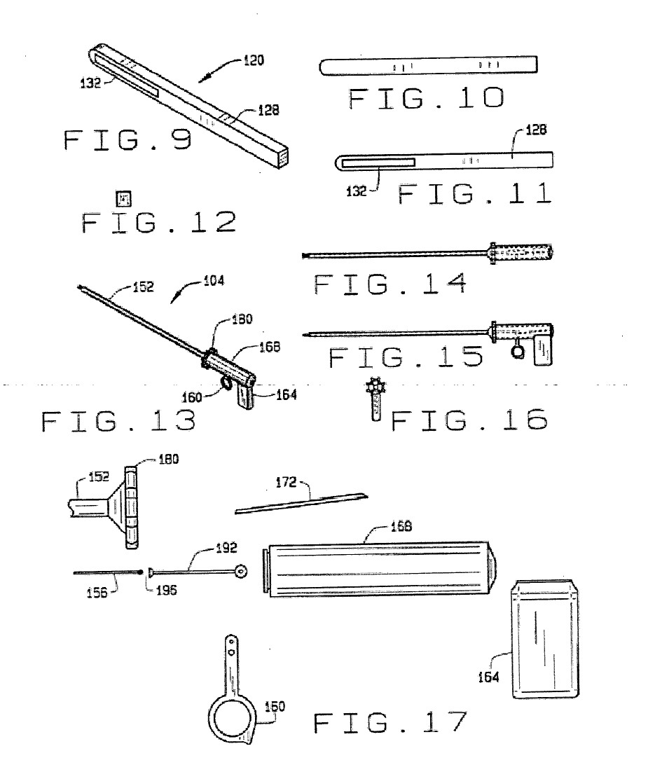

In the implementation shown in FIGS. 9-12, each electrode 120 is configured to include a generally rectangular cross-section, although other suitable shapes can also be employed.

The electrodes 120 can also include any of a wide range of tip shapes including those exemplary tip shapes shown in FIG. 5. Determining which electrode tip shape to use can include evaluating how easily tissue can be penetrated by a particular electrode tip shape, and the amount of nonspecific tissue damage caused by that penetration.

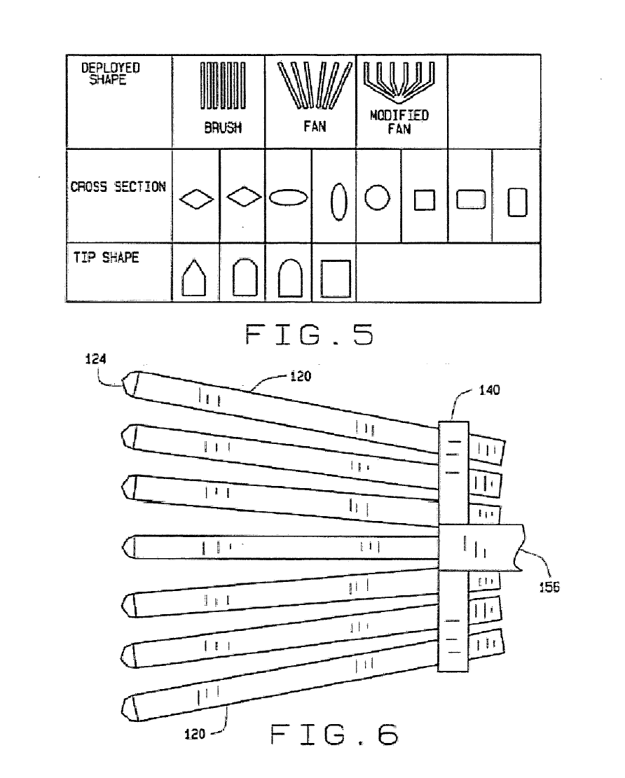

FIGS. 6 and 9-11 illustrate implementations in which the electrodes 120 each include a generally rounded tip 124 with a radius of curvature of about one-half millimeter. Alternatively, other tips shapes and sizes can be used for the electrodes 120.

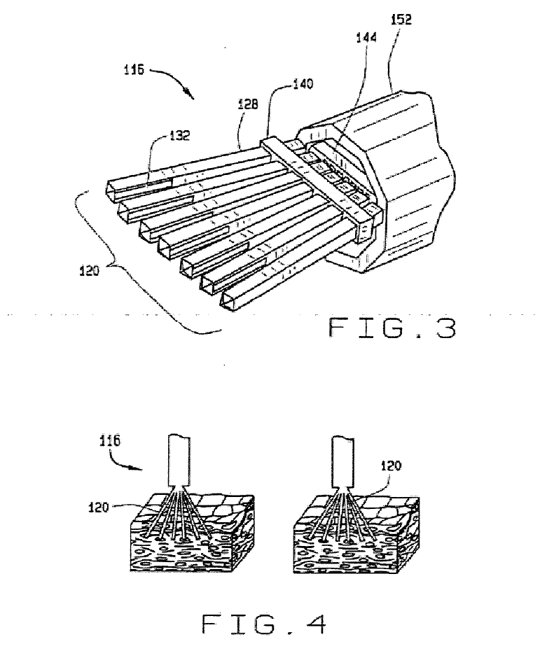

To enable greater current density between the electrodes 120, various implementations include the electrodes 120 having electrically insulative portions 128 and electrically conductive portions 132. For example, the electrodes 120 can be coated with a suitable electrical insulator (e.g., polyamide, among other electrically insulative materials). The coating can be etched using a laser to expose the underlying electrically conductive material (e.g., tungsten, etc.) of the electrodes 120. As shown in FIG. 3, the electrodes 120 include generally electrically conductive strips 132 on opposing side portions of the electrode 120. These strips 132 can be rectangular having a length of about five millimeters and a width of about one-half millimeter, although other suitable shapes and sized can be employed for the strips 132. The insulation may also be differentially applied along the length of the electrode to allow for even energy distribution despite increased distance between the electrodes at the distal end.

By using the electrode strips 132, the electrodes 120 can produce greater current density between the electrodes 120 than with completely exposed conductive regions. Further, the use of the electrode strips 132 also allows more uniform depth coagulation to be performed with minimal, or at least reduced, nonspecific tissue damage.

It should be noted that the dimensions of the electrodes 120 (and other components of the electrosurgical unit 104) may vary depending on the requirements of the particular application or surgical procedure in which the electrosurgical unit 104 will be used. For example, the dimensions may depend at least in part on the diameter of the trocar (which is a device commonly used to introduce laparoscopic instruments into the abdominal cavity). Further, the dimensions shown in the figures are for illustrated purposes only, and the invention is not limited to the particular dimensions shown in any of the figures.

In various implementations, the electrodes 120 are configured such that electrode length increases from the innermost electrode to the outermost electrodes). Accordingly, the electrode tips 124 (when the electrodes 120 are deployed) are generally aligned to allow for substantially uniform tissue depth cauterization, cutting, etc.

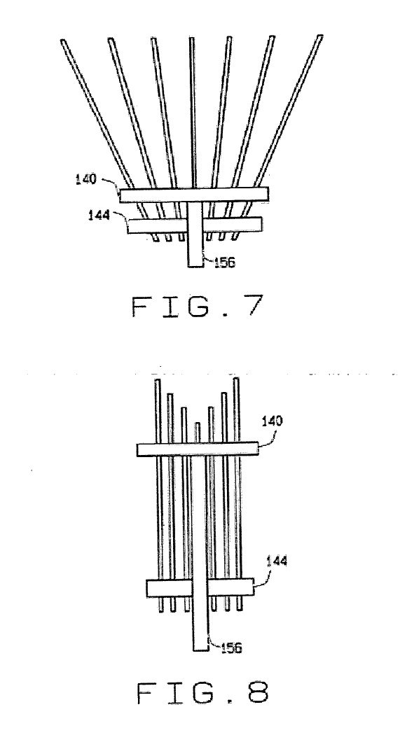

In various implementations, the electrodes 120 are movable between a collapsed or stowed position (FIG. 8) and a deployed or expanded position (FIG. 7). To allow the electrosurgical unit 104 to be inserted through a trocar, the electrodes 120 can be collapsible to a diameter less than that of the trocar. This allows the electrodes 120 to be placed laparscopically through the trocar into the patient. Once inserted through the trocar, the electrodes 120 can then be expanded which allows for safe applications of very high-density current over the multiple small areas between the electrodes, additively creating a large area of tissue that can be affected (cut, cauterized, coagulated, etc.) by the electrosurgical unit 104. It should be noted, however, that the references to trocars herein is for illustrative purposes only, and that other implementations of the invention can also be used for open surgery.

A wide range of deployment geometries can be used for the electrodes 120 including those exemplary configurations shown in FIG. 5. Electrode deployment geometry can be evaluated using various factors including homogeneity of cauterization, amount of tissue coverage, fabrication of the electrodes, etc.

As shown in FIGS. 7 and 8, the electrodes 120 can be movable between a collapsed position (FIG. 8) and an expanded position (FIG. 7). When in the collapsed position, the electrodes 120 are generally parallel within one another. But when in the expanded position, the electrodes 120 are fanned out thus increasing the tissue area that the electrosurgical unit 104 can affect, cut, cauterize, coagulate, etc. It should be understood that the electrodes also may collapse or expand sequentially or in unison when moving between the expanded position and the collapsed depending on the implementation of the device. The electrodes may also be curved to optimize surgical angles and even act as a hemostatic scooping mechanism.

By way of example only, the electrodes 120 can be sized dimensionally such that the width between the outermost electrodes is 1.30 centimeters when deployed (FIG. 7) and nine millimeters when collapsed (FIG. 8). These exemplary dimensions would thus allow the electrosurgical unit 104 to be inserted through an industry-standard 10-millimeter trocar when the electrodes 120 are collapsed, and then affect 1.30 centimeters of tissue per cycle after the electrodes 120 are deployed.

In addition, the distance separating the electrodes 120 can vary depending on the particular application or surgical procedure. For example, the electrodes 120 can be configured such that the tissue width between each pair of electrodes is consistent with industry standards.

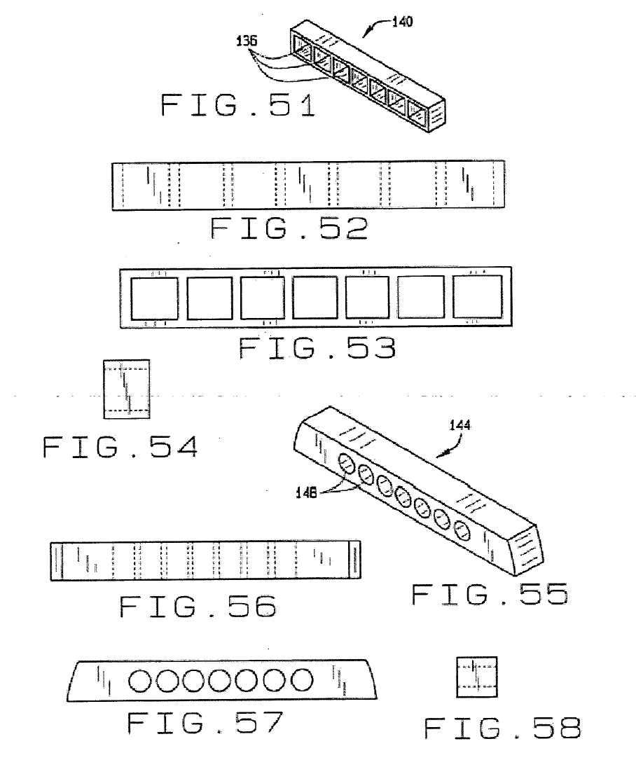

A description will now be provided of an exemplary manner in which the electrodes 120 can be caused to move between the collapsed position (FIG. 8) and the expanded position (FIG. 7). The electrodes 120 extend through openings or perforations 136 (FIGS. 51-54) in a sliding bar 140. The sliding bar 140 controls the movement of the electrodes 120 between the extended and collapsed positions.

The sliding bar 140 is translatable along the electrodes 120 between a retracted position (FIG. 8) and an extended position (FIG. 7). Retraction of the sliding bar 140 causes the electrodes 120 to expand. But when the sliding bar 140 is being extended, the sliding bar 140 causes the electrodes 120 to collapse.

As shown, the perforations 136 are preferably equally spaced along the width of the sliding bar 140. But the shape and dimensions of each perforation may vary depend on the particular electrode running through it. For example, the perforations 140 containing the outermost electrodes can be wider than the perforation containing the innermost electrodes. In an exemplary embodiment, the sliding bar 140 is made of carbon fiber to provide the sliding bar 140 with good strength and insulative properties. Alternatively, other materials can also be used for the sliding bar 140.

As shown in FIGS. 7 and 8, the electrodes 120 are generally held in place by a stationary bar 144 (FIGS. 55-58). The stationary bar 144 restricts motion at the wire-electrode interface. The stationary bar 144 defines a plurality of openings or perforations 148, through which wires may run to electrically connect to the electrodes 120.

In the illustrated embodiment, the stationary bar 144 is coupled to an inner portion of the distal end of the shaft 152 (FIG. 3). In addition, the perforations 148 are preferably equally sized and spaced along the width of the stationary bar 144 with each perforation 148 being generally circularly shaped. In an exemplary embodiment, the stationary bar 144 is fabricated from graphite, but other materials can also be used.

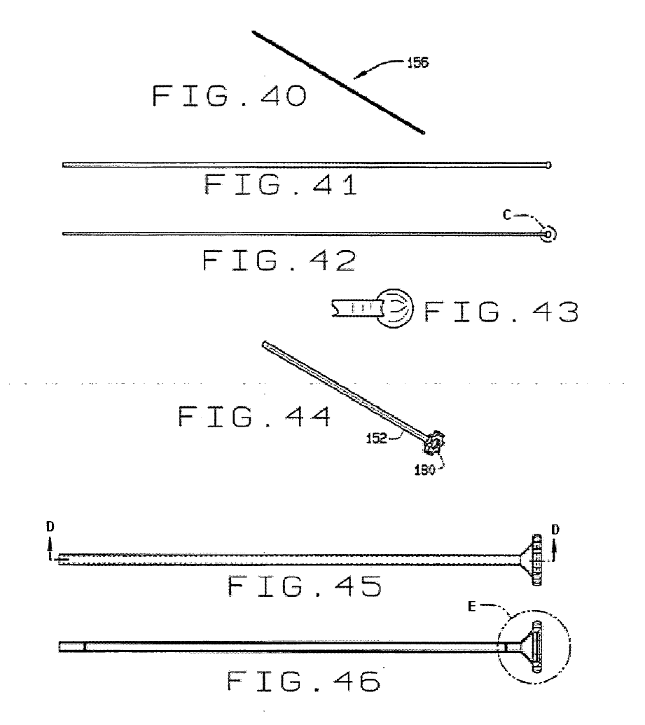

The sliding bar 140 is coupled to a main or control rod 156 (FIGS. 6-8, and 19) for common translation therewith. The main rod 156 (FIGS. 40-43) can thus be used to control movement of the sliding bar 140. The length of the main rod 156 is preferably sufficient to allow the main rod 156 to extend the length of the shaft 152 and across the top of the sliding bar 140. In an exemplary embodiment, the main rod 156 can be fabricated from graphite, although other materials can also be employed.

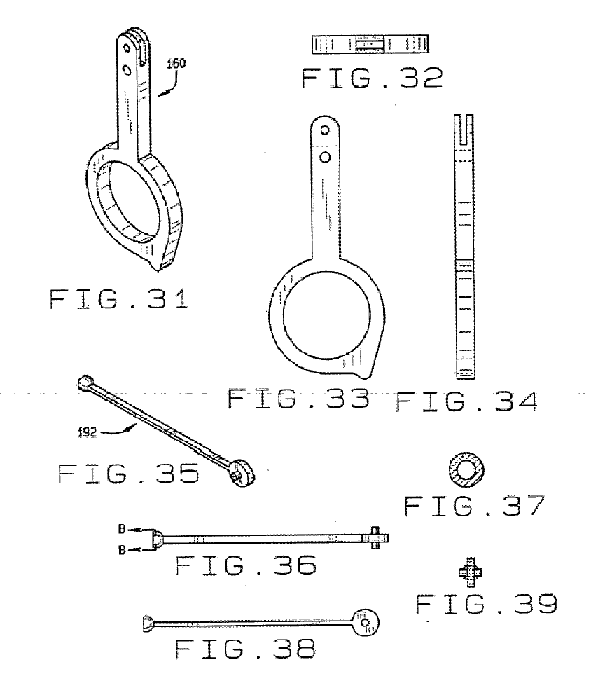

The movement of the main rod 156 (and thus the sliding bar 140 and electrodes 120) can be affected by a trigger 160 (FIGS. 31-34). An exemplary manner in which the trigger 160 can cause movement of the electrodes 120 will now be described.

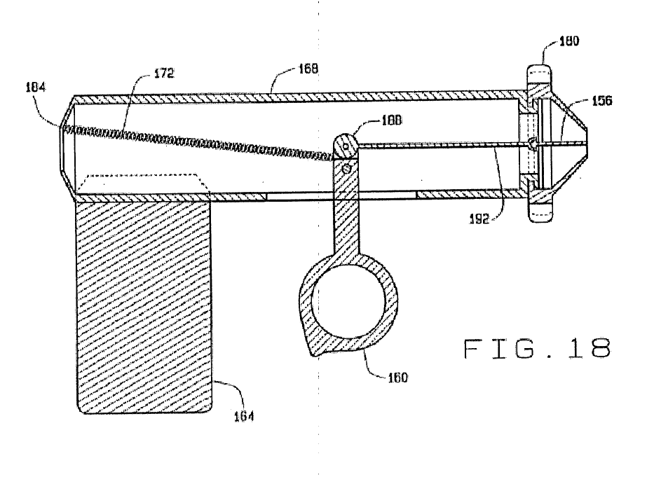

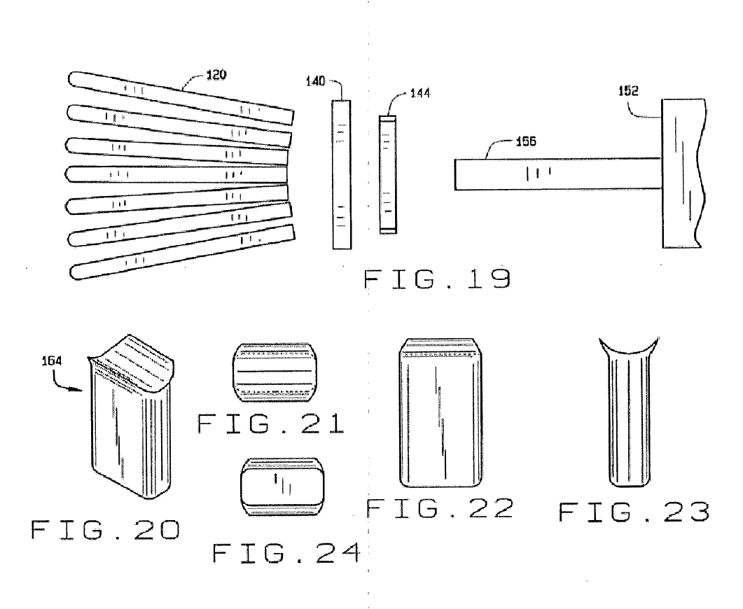

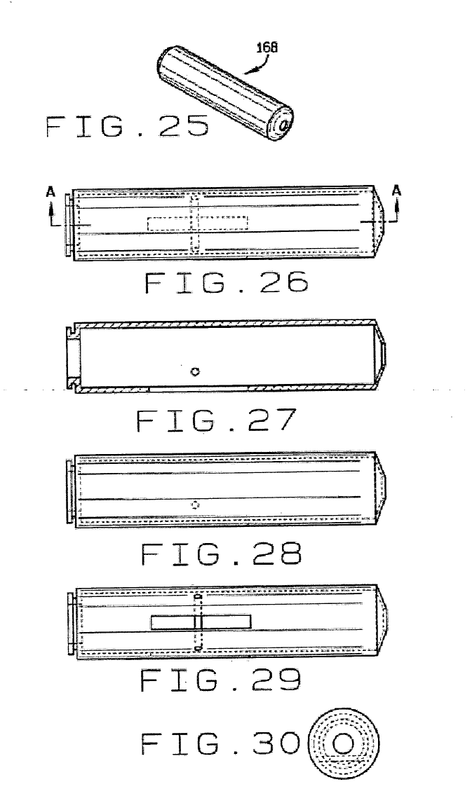

With references to FIGS. 17-18, the electrosurgical device 104 can include a handle 164 (FIGS. 20-24), and the trigger 160. Both the handle 164 and the trigger 160 are coupled to a body 168 (FIG. 18). The body 168 houses an upper half of the trigger 160, a coil spring 172, a secondary rod 192 (FIGS. 35-39), a portion of the main rod 156, and wires (not shown) electrically connecting the electrodes 120 to the switching interface or controller 108. Although the body 168 (FIGS. 25-30) is generally cylindrical in the illustrated embodiment, other shapes can also be employed.

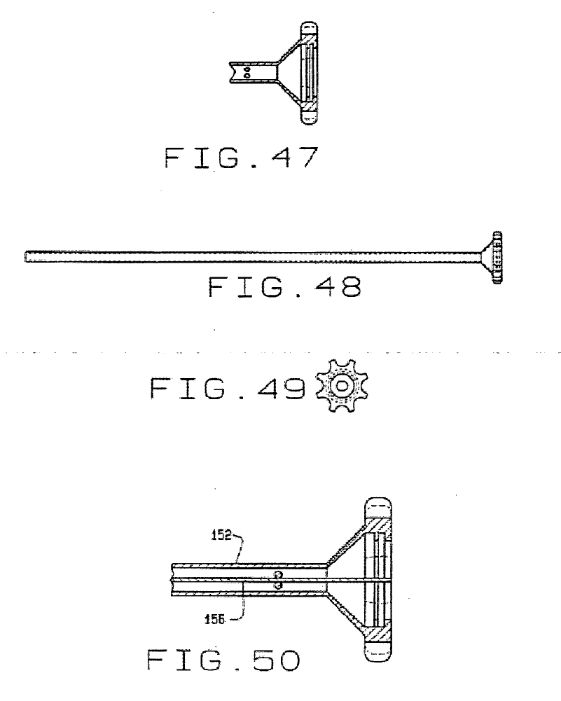

The electrosurgical device 104 also includes a shaft 152 and a knob 180 (FIGS. 44-50) for rotating the shaft 152, as shown in FIG. 1. The shaft 152 houses the main rod 156, which is involved in regulating the motion of the sliding bar 140.

As shown in FIG. 18, one end of a coil spring 172 is coupled to a back portion 184 of the body 168. The other end of the coil spring 172 is coupled to a tip or end portion 188 of the trigger 160. After squeezing the trigger 160, the tension stored in the spring 172 provides a force to restore the trigger 160, main rod 156, and sliding bar 140 back to their original positions after the trigger 160 is released. In an exemplary embodiment, the spring 172 has a k value of 0.48 pounds/inch, and is 4.32 centimeters long. Alternatively, other suitable spring configurations can also be used.

In order to pull the trigger 160, a sufficient force must be applied to overcome the biasing force of the coil spring 172 before the trigger 160 will rotate (clockwise (FIG. 18) or counterclockwise depending on the device orientation). The rotation of the trigger 160 extends the coil spring 172. It should be noted, however, that other suitable biasing devices can also be employed besides coil springs.

The end portion 188 of the trigger 160 is also coupled to a secondary rod 192. The secondary rod 192 and the main rod 156 are both coupled to a ball and socket joint 196 (FIG. 17). The ball and socket joint 196 transfers force and horizontal displacement of the secondary rod 192 to the main rod 156, without transferring the relatively small amount of vertical displacement. As shown, the ball side of the joint 196 can be attached to the main rod 156, while the socket side of the joint 196 can be attached to the secondary rod 192. Alternatively, the ball side of the joint 196 can be attached to the secondary rod 192, while the socket side of the joint 196 can be attached to the main rod 156. In an exemplary embodiment, the secondary rod 192 is fabricated out of graphite, but other suitable materials can also be employed.

Pulling the trigger 160 with sufficient force to overcome the spring biasing force causes the trigger 160 to rotate (clockwise (FIG. 18) or counterclockwise depending on the device orientation). This rotation of the trigger 160 provides force for pushing the secondary rod 192 forward (away from the user and towards the end effector 116), which, in turn, pushes the main rod 156 forward as well. The main rod 156 is connected to the sliding bar 140, which is external to the shaft 152 and generally perpendicular to the electrodes 120. The electrodes 120, running though openings 136 in the sliding bar 140, collapse when the trigger 160 is squeezed and the sliding bar 140 extends. Conversely, when the trigger 160 is released, the tension in the spring 172 returns the trigger 160 to its initial position, resulting in the backward movement of the secondary and main rods 192 and 156 towards the handle 164. In which case, the sliding bar 140 will return to its initial position near the distal end of the shaft 152 and the electrodes 120 will expand.

Referring back to FIG. 1, the electrodes 120 can be electrically connected to a controller 108. In an exemplary embodiment, wire running through the electrosurgical unit 104 is used to connect the end of each electrode 120 to the controller 108.

During operation, the controller 108 can be used to control the passage of current and voltage from electrode to electrode. For example, the controller 108 can be used to control the firing time for the multiple sets of bipolar electrodes 120 and the order in which the switching occurs between the different electrode sets. The manner in which the switching occurs and the firing time for the different electrode sets may vary depending on the particular application (e.g., type of surgery, type of tissue, etc.) in which the electrosurgical unit 104 will be used.

By way of example only, the seven linearly arranged electrodes 120 shown in FIG. 3 can be configured so as provide six bipolar electrode sets or adjacent electrode pairs. The controller 108 can be configured such that these six electrode sets fire sequentially after the end effector has been inserted through a trocar.

In various implementations, switching relays can be implemented on a breadboard and be controlled through a reprogrammable microcontroller. During an exemplary application, a surgeon can penetrate a particular portion of tissue and then press a foot pedal activating the generator 112. After this, an assistant or the surgeon can turn on the interface or controller 108 to begin the cutting, cauterization, coagulation cycle, as the case may be. In various implementations, the foot pedal will not be directly interfaced with the switching interface.

A preset firing sequence can be programmed on the controller 108 (e.g., microcontroller BS2-1C). By way of example only, various implementations can include programming and reprogramming the controller 108 for different firing times and/or sequences, as needed, using a computer software (for example Visual Basic).

In an exemplary embodiment, electrode switching is accomplished with a plurality of relays controlled through a microcontroller. The relays can be used to control the passage of current and voltage from electrode to electrode within an electrosurgical unit.

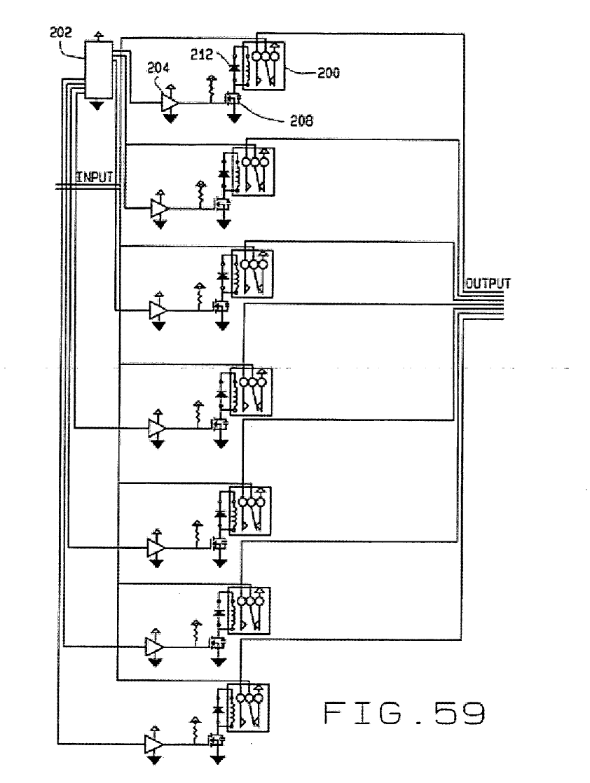

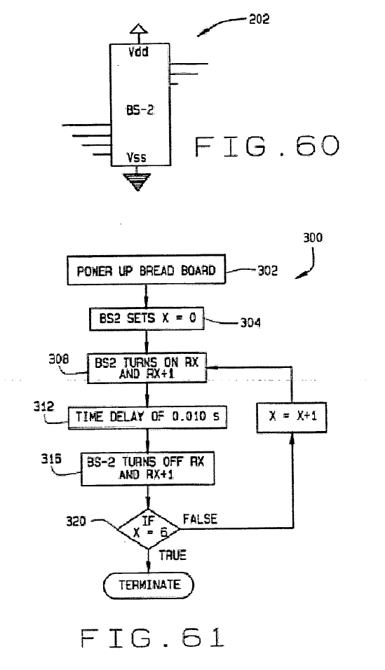

FIG. 59 illustrates an exemplary circuit diagram for an electrosurgical unit including a plurality of electrodes in which the electrodes can be controlled in accordance with a time delay sequence according to one embodiment of the invention. As shown, mechanical relays 200 are each connected to the input and to a single output. Several circuit elements have also been added to each relay 200 so as to provide power to the relay 200 and protection to the circuit and microcontroller 202. For example, a buffer 204 (e.g., SN7407N buffer) can be used to amplify the current signal from the microcontroller 202 so that devices requiring higher currents can be powered. A transistor 208 (e.g., MOSFET IRF9510 transistor) in series to the buffer 204 can be used to step up the voltage across the relay 200 so that the voltage demands of the relay 200 can be met. Further, a diode 212 (e.g., diode 1N4004G-T) can be placed next to the leads of each relay 200. This diode 212 can be involved in dissipating the voltage spike generated after closing the gate of the mechanical relay 200. The diode 212 can also dissipate changes in voltage caused by bounce when the relay 200 is powered down.

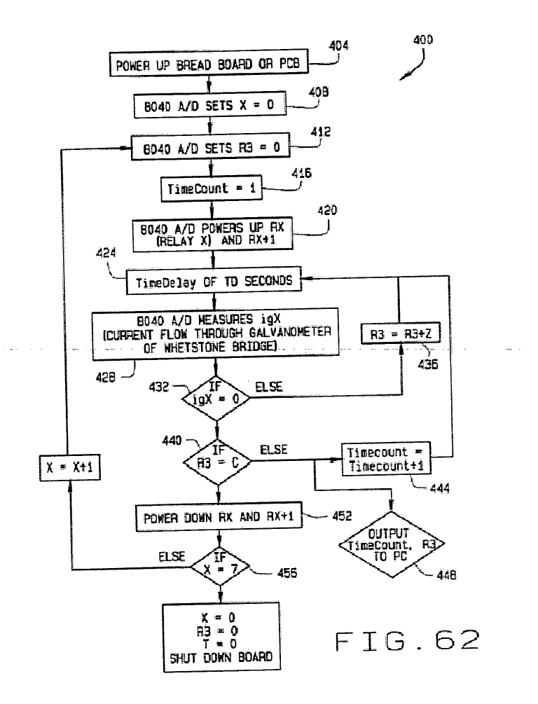

FIG. 61 is a flow diagram of an exemplary method 300 for controlling voltage and current applied to electrodes in an electrosurgical device in accordance with a timed sequence. As shown in FIG. 61, operation 302 includes activating and powering up the bread board.

Operation 304 includes the controller (e.g., BS2 microcontroller) setting a counter variable X to zero.

At operation 308, the controller activates a first set of adjacent electrodes, such as electrodes 1 and 2 in FIG. 8.

Operation 312 includes a time delay, such as 0.010 seconds, to allow current to pass through the tissue and start cutting, cauterizing, coagulating, etc. It should noted, however, that the time delay can vary depending on the particular application and various factors such as the type of surgical procedure, type of tissue being affected (e.g., cut, cauterized, coagulated, etc.), voltage and current being applied to the tissue, tissue impedance, tissue temperature, electrode configuration (e.g., size, shape, number of, and deployment geometry of the electrodes), among other factors.

Operation 316 includes the controller turning off or deactivating the currently activated electrode set or pair.

At operation 320, a determination is made as to whether each electrode pair has been activated. If so, then the present cycle is completed. Otherwise, the method 300 returns to operation 308. The number of times that operations 308 through 320 are repeated can depend on the number of adjacent electrode pairs that the electrosurgical unit includes.

Accordingly, the exemplary method 300 enables switching between the different electrode sets to be controlled in accordance with a timed delay, and with the electrode sets being deployed sequentially from one end of the linear array to the other end. By selectively activating the adjacent electrode pairs in this exemplary manner, an electrosurgical unit can incrementally apply relatively high density current in relatively small increments over a larger area.

FIG. 62 forms a flow diagram of an exemplary method 400 for controlling operation of the electrodes 120 depending on the impedance caused by the tissue through which electrical current is flowing. As shown in FIG. 62, operation 404 includes activating and powering up the bread board.

Operation 408 includes the controller (e.g., 8040 A/D controller) setting a counter variable X to zero.

Operation 412 includes the controller setting the Wheatstone bridge resistor variable R3 to zero.

Operation 416 includes setting TimeCount to one.

Operation 420 includes the controller activating the relays for a first set of adjacent electrodes such that one of the electrodes is active while the other is the passive or return electrode.

Operation 424 includes a time delay (TD). This time delay allows the impedance of the tissue (and therefore unknown resistor in the Wheatstone bridge) to change before the current is measured again at operation 428, described below. The time delay can vary depending on the particular application and various factors such as the type of surgical procedure, type of tissue being affected (e.g., cut, cauterized, coagulated, etc.), voltage and current being applied to the tissue, electrode configuration (e.g., size, shape, number of, and deployment geometry of the electrodes), among other factors.

During the time delay at operation 424, current can pass through the tissue and cut, cauterize, coagulate, etc. the tissue. But in various implementations, a majority of the cauterization does not occur through this time delay. Instead, a majority of the cauterization occurs while the loop (formed by operations 424-428-432-436-424) runs until the current flow (igx) through the galvanometer of the Wheatstone bridge reaches zero. The time of cauterization therefore depends on how many times the loop (operations 424-428-432-436-424) must run before igx reaches zero, and then again on how many times the larger loop or process (formed by operations 424-428-432-440-444-424) must occur (allowing the current to reach zero) before the impedance reaches a desired value.

Operation 428 includes the controller measuring the current flow through the galvanometer of the Wheatstone bridge.

At operation 432, a determination is made as to whether igx (the current flow through the galvanometer of the Wheatstone bridge) is zero. If the igx is not zero, then operation 436 includes adding an adjustment factor Z to the Wheatstone bridge resistor R3. The adjustment factor Z may prove useful for testing and evaluation purposes. But it should be noted that the adjustment factor Z will not be necessary or included in all implementations of the invention.

As shown in FIG. 62, if igx is determined to be zero, then operation 440 includes determining whether R3 is equal to C. Determining when R3=C represents when the tissue is adequately cauterized/cut/coagulated, the C constant is determined by the impedance of the tissue at this state. The C constant will vary depending on type of tissue and whether that tissue is being cut, coagulated, cauterized, etc.

If R3 is not equal to C, operation 444 includes setting Timecount=Timecount+1, and then returning to operation 424.

Operation 448 includes optionally recording output, TimeCount, R3, for example, for evaluation purposes.

If R3 is determined to be equal to C at operation 440, then the method proceeds to operation 452 in which the controller turns off and deactivates the current electrode set.

At operation 456, a determination is made as to whether each electrode pair has been used. If so, then the present cycle is completed. But if not, then the method returns to operation 412. The number of times that operations 412 through 456 are repeated can depend on the number of adjacent electrode pairs that the electrosurgical unit has.

Accordingly, the exemplary method 400 enables controllable switching that is dependent on the impedance of the tissue to which electrosurgical energy (e.g., RF energy) is being applied. By way of background, heat can be produced as the electrons flowing through the patient's tissue overcomes the impedance associated with that tissue.

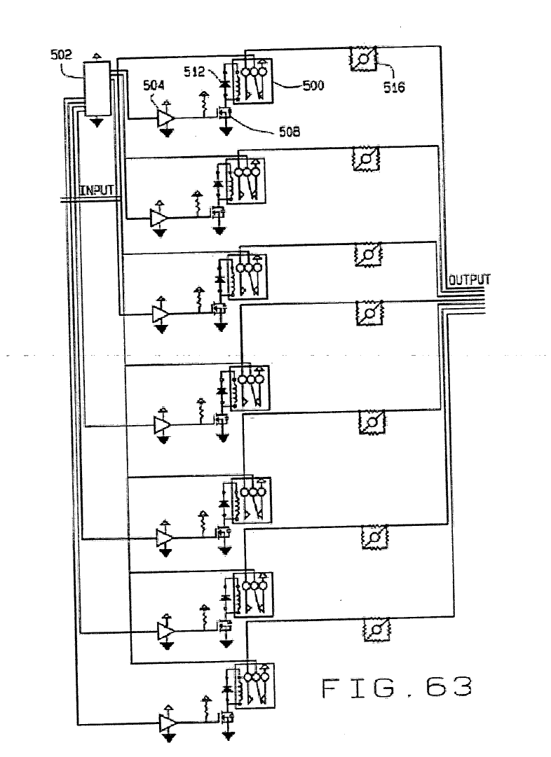

FIG. 63 is an exemplary circuit diagram for an electrosurgical unit including a plurality of electrodes in which the electrodes be controlled in accordance with impedance feedback according to one embodiment of the invention. As shown, each of the relays 500 is connected to the input and to a single output. A controller 502 can be connected to a voltage source from Ground to power components, MIC, and Bridges. The circuit also includes buffers 504 that can be used to amplify the current signal from the microcontroller 502 so that devices requiring higher currents can be powered. Transistors 508 each in series to a corresponding buffer 504 can be used to step up the voltage across the corresponding relay 500 so that the voltage demands of that relay 500 can be met. Further, a diode 512 can be placed next to the leads of each relay 200. Each diode 512 can be involved in dissipating the voltage spike generated after closing the gate of the corresponding relay 500. Each diode 512 can also dissipate changes in voltage caused by bounce when its corresponding relay 200 is powered down.

With further reference to FIG. 63, the circuit further includes Wheatstone bridges 516 each having a voltage source (the voltage source is not shown in FIG. 63). The bridges 516 have varied resistance. During operation of the electrosurgical unit, these bridges 516 can be used to determine impedance of the tissue being cauterized or otherwise affected. For each bride 516, the value of Rx is equal to (R2/R1)*R3, where R1 and R2 are preset values given by the manufacture of the bridge, and R3 is a variable resistor and is given by the resistor with the line drawn across it.

Another aspect of the invention is the manner in which the various electrosurgical units are able to act and affect tissue. Existing electrosurgical units generally interact with tissue by clamping on the tissue thus limiting their applicability to vessels and relatively small, thin pieces of tissue. But in various implementations of this invention, electrosurgical units actually penetrates the tissue being affected, cauterized, cut, coagulated, etc. For example, an electrosurgical unit of the present invention can cauterize in a different plane than those devices that clamp. An electrosurgical unit of the present invention is capable of acting through a depth in the tissue, thereby allowing the electrosurgical unit to be used on relatively large sections of tissue and organs, not limited by size or geometry. This, in turn, can enable surgical procedures to be performed such as removing entire sections of an organ (for example a partial nephrectomy), as opposed to merely clamping closed a duct or blood vessel, to which current devices are limited.

Various implementations of the electrosurgical unit can also include a mechanical effect to the tissue in addition to the effect of the electrosurgical energy. A mechanism for imparting pressure and/or tissue cutting mechanism may also be incorporated to optimize the effect of the device. For example, a blade for mechanically cutting may be employed to adapt the electrosurgical unit to a specific application.

Touch screen interfaces for controlling generator settings, such as cutting/cauterization/coagulation time, power, and output waveform, can also be implemented and added to electrosurgical systems of the present invention. In addition, the timing settings and firing sequences could also be changed for different tissues so that each can be more efficiently cauterized and/or cut with a lower degree of nonspecific tissue damage. It is also possible to reduce the shaft length of an electrosurgical unit to allow it to be used in open surgery, or an oral surgical field, among other surgical disciplines.

By utilizing multiple bipolar circuits in a single instrument, various implementations of the present invention can increase the efficacy of cauterization, enable cauterization and bloodless cutting of tissue through depth, and allow safe application of relatively high density current in small increments over a larger area. Various implementations can also enable depth coagulation without a great deal of nonspecific tissue damage over a generous area, than that which is currently being done with bipolar needles.

By implementing sequential firing of bipolar electrode sets, various implementations also allow for safe application of relatively high density current in small increments over a larger area.

The novel concepts embodied in the various implementations of this invention will allow physicians greater flexibility in procedures involving electrosurgery. The time per procedure will also be reduced, and the targeted region of tissue will also be more precise. Further, the generator interface can also allow existing electrosurgical generators to be used with the various electrosurgical units of the present invention.

Procedures in many medical fields ranging from oral, craniofacial surgery, gynecology, urology, general surgery to other surgical and non-surgical disciplines will also be directly impacted by the use of one or more of the implementations of this invention. Indeed, various implementations of the present invention can be used in a wide range of electrosurgical applications and surgical disciplines for cutting, cauterizing, coagulating, combinations thereof, etc. Exemplary applications can include, but are not limited to, open surgery, laparoscopy and other types of minimally invasive surgery, parenchymal transaction, endoscopy applications, craniofacial applications, periodontal applications, veterinarian surgical procedures, among others.

Some more specific examples will now be provided of possible uses for one or more of the various implementations of the present invention. In Urology, an electrosurgical device of the present invention might be used for a partial nephrectomy to cut and remove a diseased portion of the patient's kidney. In General Surgery, an electrosurgical device of the present invention might be used for a partial hepatectomy to remove a diseased portion of the patient's liver. And, in Obstetrics and Gynecology (OB/-GYN), an electrosurgical device of the present invention might be used for a parenchymal transection of the patient's uterus to remove diseased components.

It should be noted that the dimensions, materials, shapes, and configurations of the various components described herein may vary depending on the requirements of the particular application in which an electrosurgical device will be used. The various dimensions, materials, shapes and configurations shown in the figures are for illustrative purposes only.

When describing elements or features of the present invention or embodiments thereof, the articles “a”, “an”, “the” and “said” are intended to mean there are one or more of such elements or features. The terms “comprising”, “including” and “having” are intended to be inclusive and mean there may be additional elements or features beyond those specifically described.

Those skilled in the art will recognize that various changes can be made to the exemplary embodiments and implementations described above without departing from the scope of the present invention. Accordingly, all matter contained in the above description or shown in the accompanying drawings should be interpreted as illustrative and not in a limiting sense.

What is claimed is:

1. An electrosurgical device comprising a plurality of electrodes for forming a plurality of bipolar circuits usable for affecting a patient's tissue during a surgical operation, at least one of said electrodes being operable as an active electrode in one of said bipolar circuits and as a return electrode in another one of said bipolar circuits.

2. The electrosurgical device of claim 1, wherein the electrodes are arranged in a linear array.

3. The electrosurgical device of claim 2, wherein the electrodes are configured such that electrode length increases from the innermost electrode to the outermost electrodes of the linear array.

4. The electrosurgical device of claim 1, wherein the electrosurgical device includes seven electrodes.

5. The electrosurgical device of claim 1, wherein each said electrode includes an electrically insulative coated portion and at least one uncoated electrically conductive portion.

6. The electrosurgical device of claim 5, wherein at least one electrode includes a pair of uncoated electrically conductive portions on opposing side portions of the electrode.

7. The electrosurgical device of claim 1 further comprising a tissue cutting mechanism for mechanically affecting tissue.

8. The electrosurgical device of claim 7 wherein the tissue cutting mechanism is a blade.

9. The electrosurgical device of claim 1, further comprising an electrode deployment device for moving the electrodes between a collapsed position in which the electrodes are generally parallel within one another, and an expanded position in which the electrodes are fanned out.

10. The electrosurgical device of claim 1, wherein each said electrode includes a generally rectangular cross-section with a generally rounded tip.

11. The electrosurgical device of claim 1, wherein the electrodes are configured to penetrate the patient's tissue thereby enabling the device to act through a depth in the tissue.

12. The electrosurgical device of claim 1, wherein the electrodes are configured to move between a collapsed position and an expanded position in which the electrode tips are generally aligned with one another, thereby allowing the device to affect the tissue at a substantially uniform tissue depth.

13. An electrosurgical device comprising a plurality of electrodes, including a plurality of adjacent electrode pairs, and a controller for selectively activating the adjacent electrode pairs to incrementally affect tissue including a first tissue portion and a second tissue portion, whereby activating a first electrode pair affects the first tissue portion, and activating a second electrode pair affect the second tissue portion.

14. The electrosurgical device of claim 13, wherein the controller is configured to active adjacent the electrode pairs sequentially.

15. The electrosurgical device of claim 13, wherein each said electrode includes an electrically insulative coated portion and at least one uncoated electrically conductive portion.

16. The electrosurgical device of claim 15, wherein each said electrode includes an electrically insulative coating that is etched to expose the at least one electrically conductive portion.

17. The electrosurgical device of claim 15, wherein each said electrically conductive portion is generally rectangular shaped.

18. The electrosurgical device of claim 15, wherein the electrodes comprise tungsten, and the insulative coating comprises polyamide.

19. The electrosurgical device of claim 13, further comprising: a stationary member coupled to an end portion of each said electrode to inhibit movement of the electrode end portions; and a sliding member defining a plurality of apertures through which the electrodes extend, the sliding member being slidable along the length of the electrodes to move the electrodes between a collapsed position in which the electrodes are generally parallel with one another and generally perpendicular to the sliding member, and an expanded position in which the electrodes are fanned out.

20. The electrosurgical device of claim 19, further comprising a trigger for causing the sliding member to move between an extended position in which the electrodes are in the collapsed position, and a retracted position in which the electrodes are in the expanded position.

21. The electrosurgical device of claim 13, further comprising a Wheatstone bridge to monitor impedance from tissue through which electrical current is flowing.

22. The electrosurgical device of claim 21, wherein the controller is configured to selectively activate adjacent electrode pairs in connection with impedance as monitored by the Wheatstone bridge.

23. The electrosurgical device of claim 13, wherein the controller includes a plurality of mechanical relays each associated with a corresponding one of said electrodes.

24. The electrosurgical device of claim 13, wherein the controller includes a plurality of mechanical relays each associated with a corresponding one of said electrodes to enable switching between the bipolar circuits.

25. The electrosurgical device of claim 13, further comprising a generator for powering the electrosurgical device.

26. The electrosurgical device of claim 13, wherein each said electrode includes a generally rectangular cross-section with a generally rounded tip.

27. The electrosurgical device of claim 13, wherein the electrodes are configured to penetrate the patient's tissue thereby enabling the device to act through a depth in the tissue.

28. An electrosurgical device comprising a plurality of electrodes, wherein a section of each electrode is intended to make contact with a patient's tissue, the section includes an electrically insulative portion and an electrically conductive portion.

29. The electrosurgical device of claim 28, wherein each said electrically conductive portion is generally rectangular shaped.

30. The electrosurgical device of claim 28, wherein at least one electrode includes a pair of uncoated electrically conductive portions on opposing side portions of the electrode.
